(19) United States
(12) Patent Application Publication
(10) Pub. No.: US 2004/0234992 A1
(43) Pub. Date: Nov. 25, 2004

(54) Method of detection of conformational change in a nucleic acid duplex by treatment with oxidising or reactive agent as a result of exposure to environmental or chemical conditions

(76) Inventors: Richard G Cotton, Kooyong (AU); Chinh Thien Bui, Braybrook (AU); Abdulkerim Bedir, Samsun (TR)

Correspondence Address: GREENLEE WINNER AND SULLIVAN P C, 5370 MANHATTAN CIRCLE, SUITE 201, BOULDER, CO, 80303, US

(21) Appl. No.: 10/489,472

(22) PCT Filed: Sep. 13, 2002
(86) PCT No.: PCT/AU02/01259

(30) Foreign Application Priority Data
Sep. 13, 2001 (AU) PR 7673

(51) Int. Cl. C12Q 1/68

(52) U.S. Cl. 435/6

(57) ABSTRACT

A method of detecting a conformational change in a nucleic acid sample by treatment with oxidising agent for example KmnO₄ to determine difference in formation or rate of formation of reaction product MnO₂ or consumption or rate of consumption of KmnO₄, which indicates conformational change in the test sample, in particular when the DNA is exposed to an environmental salt concentration and temperature or electrical current. The method investigates conformational change in the DNA, for example when DNA is exposed to an intercalating agent. The use of this method for investigating chemical compounds for conformational change in the DNA is also described. The conformational change is determined by colorimetric inorganic assay, by measuring absorbance by means of a spectrophotometer. The use of other oxidising agents besides permanganate comprising OsO₄ or chromic acid or ozone gas, or peroxides or perbenzoic acids or electrical current, are described in the body of the specification.

### FIELD OF THE INVENTION

The present invention relates to methods for detection and quantification of conformational changes in duplex nucleic acids. In particular, but not exclusively, the invention relates to methods which may be utilised to test the effects on duplex nucleic acid conformation of environmental conditions and exposure to chemical compounds.

### BACKGROUND OF THE INVENTION

DNA conformation and its perturbation by small organic and inorganic molecules or environmental factors is an issue of importance for research, in relation to toxicity testing of chemical compounds and other environmental agents and particularly to the pharmaceutical, food and cosmetic industries, among others. Many advanced methods have been developed to monitor the interaction between duplex nucleic acids (such as DNA and RNA) and environmental factors or chemical compounds (such as intercalators, other DNA-binding ligands or high concentrations of salts such as NaCl, MgCl₂, CaCl, etc.) in an endeavour to understand conformational changes, binding energies and kinetics of the resulting adducts.^{1,2}

Some physical and chemical methods utilised in the past to monitor conformational changes include sedimentation measurement, fluorescent energy transfer experiments, foot-printing assays, X-ray crystallography, nuclear magnetic resonance (NMR), polymerase chain reaction (PCR) and enzymatic³ and chemical cleavage. While these methods are powerful, they are generally time consuming, require sophisticated equipment and skilful operators. Other biological testing means such as the use of laboratory animals, plants such as the Tradescantia plant (to detect radiation) and bacterial or mammalian cell lines have also been adopted in the past in an endeavour to determine likely mutagenicity or carcinogenicity of chemical compounds or environmental conditions. The Ames test which utilises salmonella bacteria is probably the best known of these tests presently available. Unfortunately, such biological means of testing do not provide an accurate predictor of likely nucleic acid conformational change in nucleic acids from the organism of interest and are often time consuming and expensive to conduct.

There is therefore a pressing need to provide simple and accurate means of detecting and/or quantifying conformational changes in duplex nucleic acids which may result from environmental conditions or exposure to chemical compounds. Preferably such means avoid the need to culture bacterial cells and avoid the use of a separation step, or alternatively, provides greater resolution or discrimination (ie enhanced separation) in methods relying on a separation step.

### SUMMARY OF THE INVENTION

The present invention relates generally to a method of detecting a conformational difference between two nucleic acid duplexes or a conformational change in a first or test nucleic acid duplex as a result of exposure to chemical or environmental conditions. The methods of the invention can be performed by: (i) treating a first or test nucleic acid duplex with an effective amount of an oxidising or reactive agent for a time and under conditions sufficient to oxidise or react with a perturbed base or bases in the first or test nucleic acid duplex; (ii) monitoring the formation, or rate thereof, of one or more reaction products and/or the consumption, or rate thereof, of one or more starting agents; and (iii) determining if there is a difference in the formation, or rate thereof, of one or more reaction products and/or the consumption, or rate thereof, of one or more starting agents between the first or test nucleic acid duplex and that of a second or control nucleic acid duplex which has separately been subjected to the same conditions of steps (i) and (ii). A difference in the formation, or rate thereof, of one or more reaction products and/or the consumption, or rate thereof, of one or more starting agents between the first or test nucleic acid duplex and the second or control nucleic acid duplex is taken to be indicative of a conformational difference between the two duplexes or a conformational change in the first or test nucleic acid duplex.

Thus, according to one embodiment of the invention there is provided a method of detecting a conformational change in a test nucleic acid duplex sample, which has been exposed to test conditions, relative to a control nucleic acid duplex sample, which comprises the steps of separately subjecting the test and control duplex samples to treatment with an oxidising or reactive effective amount of an oxidising or reactive agent for a time and under conditions sufficient to oxidise or otherwise react with perturbed bases within the duplexes, and determining if there is a difference in:

(a) formation or rate of formation of one or more reaction products; and/or

(b) consumption or rate of consumption of one or more starting agents;

between test sample and control sample, wherein a difference indicates conformational change of the test sample.

The present invention may also be useful in determining the degree of unwinding of a nucleic acid molecule (ie extent of conformational change) exposed to test conditions.

Thus, in another embodiment of the invention there is provided a method of determining extent of conformational change in a nucleic acid duplex sample, which has been exposed to test conditions, relative to a control nucleic acid duplex sample, which comprises the steps of separately subjecting the samples to treatment with an oxidising or reactive effective amount of an oxidising or reactive agent for a time and under conditions sufficient to oxidise or otherwise react perturbed bases within the duplexes, and quantifying difference in:

(a) formation or rate of formation of one or more reaction products of oxidisation or reaction; and/or

(b) consumption or rate of consumption of one or more starting agents of oxidisation or reaction; between test sample and control sample and comparing the difference to quantified differences determined from exposing like nucleic acid duplex samples from testing with agents that give rise to a known extent of conformational change.

In another embodiment, the invention provides a method of detecting a conformational difference between two nucleic acid duplex samples which comprises the steps of separately subjecting first and a second nucleic acid duplex samples to treatment with an oxidising or reactive effective amount of an oxidising or reactive agent for a time and under conditions sufficient to oxidise or otherwise react with perturbed bases with the duplexes and determining if there is a difference in:

(a) formation, or rate of formation, of one or more reaction products; and/or

(b) consumption, or rate of consumption, of one or more starting agents;

between the first and second nucleic acid duplex samples wherein a difference is indicative of a conformational difference between the two nucleic acid duplex samples.

In yet another embodiment of the invention, there is provided a method of determining whether a test nucleic acid duplex has been exposed to conformational changing conditions comprising the steps of separately subjecting the test nucleic acid duplex sample and a control nucleic acid duplex sample to treatment with an oxidising or reactive effective amount of an oxidising or reactive agent for a time and under conditions sufficient to oxidise or otherwise react with perturbed bases within the duplexes, and determining if there is a difference in:

(a) formation or rate of formation of one or more reaction products; and/or

(b) consumption or rate of consumption of one or more starting agents;

between the test duplex sample and control duplex sample, wherein a difference indicates exposure of the test sample to conformational changing conditions.

In one embodiment of the invention the test nucleic acid duplex sample is exposed to an environmental condition such as radiation, change in temperature, change in pH, electrical current, magnetic field or change in salt concentration in an endeavour to determine whether such environmental conditions give rise to conformational change.

In another embodiment of the invention the test nucleic acid duplex sample is exposed to one or more chemical compounds in an endeavour to determine whether such compounds give rise to conformational change which may lead to mutagenicity, carcinogenicity or cell death.

For example, the chemical compounds being tested may be organic or inorganic and may include compounds potentially useful in pharmaceutical or veterinary products, in products for human or animal consumption, in cosmetics or other personal use products (eg shampoos, soaps, deodorants, hair dyes, sunscreens), in clothing (eg dyes, sizing agents, mothproofing agents, shrink resistance agents), in manufacturing processes or they may be compounds identified from sources such as plant, animal or microorganism extracts or soil, water or air samples.

In another preferred embodiment of the invention the oxidising agent may be selected from KMnO₄, OSO₄, chromic acid, ozone gas, peroxides, perbenzoic acids and electrical current and the reactive agent from hydroxylamine, carbodiimide and enzymes.

In a still further embodiment of the invention the determination or quantification of a difference in (a) formation or rate of formation of one or more reaction products of oxidisation or reaction; and/or (b) consumption or rate of consumption of one or more starting agents of oxidisation or reaction may be conducted utilising spectroscopy (eg UV visible), chromatography, titration, colorimetry, melting point determination, electrical current, coupling of oxidised or reduced species to another agent, use of a redox stain or visual detection.

In one preferred embodiment of the invention a difference in formation of MnO₂ between the test sample and control sample is detected and/or quantified in the situation where KMnO₂ is the oxidising agent, by colorimetric inorganic assay. Preferably, this assay involves measuring absorbance at between about 400 nm and about 440 nm, preferably at about 420 nm. Preferably the oxidation in this embodiment of the invention is carried out in a solution of TEAC or TMAC.

### BRIEF DESCRIPTION OF THE FIGURES

The invention will be described further, by way of example only, with reference to the figures wherein:

FIG. 1 shows correlation curve between the permanganate oxidation level (Log₁₀A420 nm) and the unwinding degree of calf thymus DNA induced by intercalators. The intercalator (10 μl, 6.3 mmol) was incubated with the solution containing calf thymus DNA (5 μl, 8.27 μg) and NaCl (600 μl, 5M NaCl solution) in water (355 μl) at 40° C. for 1 h. The reaction mixture was treated with KMnO₄ (30μ of 10 mM solution) and maintained at 35° C. for 60 min. The absorbance at 420 nm was obtained at 2 min and 60 min by Cary 300 spectrophotometer (Varian Inc.). The level of oxidation was based on the absorbance at A420 nm of the experiment and control (without DNA) experiments as described in the experimental section. Four intercalators (with published data on unwinding degree) were used in the experiment: doxorubicin (10°), daunomycin (12°), 9-aminoacridine (17°) and ethidium bromide (28°); and

FIG. 2 shows correlation curve between the permanganate oxidation level (A420 nm) and different concentrations of ethidium bromide (nmol). Calf thymus DNA (5 μl, 8.27 μg) was incubated with ethidium bromide (4 different concentrations were used: 1.575, 3.15, 6.3 and 12.6 nmol) at 40° C. for 1 h. The reaction mixture was treated with 35 μl of 10 mM KMnO₄ solution at 35° C. Absorbance of the mixture was obtained at 2 and 60 min by the Cintra-10 spectrophotometer (GBC). The level of oxidation was based on the absorbance at A420 nm of the experiment & control experiments (without DNA) as described in the experimental section.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that the prior art forms part of the common general knowledge in Australia.

As indicated above, there are numerous purposes in relation to which the present invention has utility. For example, the invention may be useful in determining the effect on nucleic acid duplex conformation of radiation (such as electromagnetic radiation or ionising radiation), temperature change, pH change, electronic current, magnetic field or change in salt concentration, or for example the impact on conformation of the nucleic acid duplex of a chemical compound. Such chemical compounds may be synthetic or naturally derived, may be proposed for use in pharmaceuticals, veterinary products, agrochemicals, human or animal consumables, cosmetics or other personal use products, in clothing or in manufacturing processes or the compounds may be released into the environment from a manufacturing or industrial process and may for example be identified from plant, animal or microorganism extracts or soil, water or air samples. Chemical compounds envisaged include enzymes, such as polymerase enzymes and known DNA binding agents such as ethidium bromide, doxorubicin, daunomycin and 9-aminoacridine. Particularly in the cases of potential pharmaceutical or veterinary products, agrochemicals (eg fertilizers, pesticides, herbicides), human or animal foodstuffs and cosmetics it is important to establish whether such compounds may give rise to changes in nucleic acid conformation.

Such changes may be indicative of potential mutagenicity, carcinogenicity or cell death. It is possible in the case of pharmaceutical or veterinary products or antimicrobial or agrochemical products for it to be desirable to bring about nucleic acid duplex conformational changes which may be deleterious to cells or organisms comprising the duplex nucleic acids. In particular, agents which are deleterious to cells (result in cell death) may be useful as anticancer or antimicrobial, especially antibacterial, agents.

In another aspect of the invention, and where the extent of conformational change caused by a compound is known, the present methods may be utilised to determine concentration of the compound.

By the phrases “conformational change” or “changes in conformation” (and variations thereof) of a nucleic acid duplex it is intended to convey alterations in the relative spatial arrangement of components of the duplex and particularly the bases, relative to their position in the control nucleic acid duplex. The control nucleic acid duplex is preferably a nucleic acid duplex or sample thereof which is substantially identical to the nucleic acid duplex or sample thereof which is being tested for conformational change. In the case of one or more bases which have spatial position altered relative to the corresponding position in the control duplex, such bases may be referred to as “perturbed bases”. Duplex nucleic acids including one or more perturbed bases may be referred to as “perturbed duplex”. A “conformational difference” between two nucleic acid duplexes is intended to mean a difference in the relative spatial arrangement of the components of the duplex, in particular the bases.

As will be well understood by persons skilled in the art the usual conformation of duplex DNA is the B conformation which takes the form of a right-handed double helix defined by major and minor grooves and wherein all of the bases are in the anti-conformation. A second, higher energy conformation of DNA is the Z-conformation wherein there are two strands of anti-parallel DNA joined by Watson-Crick base pairing and wherein the bases alternate between the anti- and unusual syn-conformation. Within Z-DNA the backbone follows a zigzag path and there is only a single narrow groove, which corresponds to the minor groove of B-DNA. In the case of B-DNA which is exposed to high salt concentration (for example in excess of 3M NaCl, CaCl₂, MgCl₂ etc.), the B-form is transformed into the Z conformation. There are many other common conformations of duplex DNA and innumerable minor modifications of conformation which may result from environmental conditions or exposure to chemical compounds. In particular, it is well understood that DNA binding agents, and especially intercalating agents give rise to conformational changes. In the case of intercalating agents which often take the form of planar aromatic structures, there may be an induction of unwinding within the duplex structure, which can readily be detected by the methods according to the present invention. In contrast to the intercalating agents DNA major- or minor-groove binding ligands generally give rise to only minor conformational change.

As used within the detailed description, the term “modification” (and variants thereof) can be used interchangably with “oxidation” and “reaction” (and variants thereof).

As used herein, a “nucleic acid duplex” refers to a duplex arising from two single stranded nucleic acid molecules hybridised together. Preferably the nucleic acid duplex is fully complementarily base-paired although it will be appreciated that the duplex may contain one or more mismatched or unmatched bases. Complementary base pairing occurs in a double stranded nucleic acid duplex consisting of a first single stranded nucleic acid molecule hybridised together with a second single stranded molecule when G and C bases bind together and A and T bases bind together (or U and A bind together). In order to ensure that conformational changes and not base changes, ie mutations, are being determined, the test and control nucleic acid duplexes will possess substantially identical nucleic acid sequences, whether the two nucleic acid duplexes are fully base paired or contain one or more mismatched or unmatched bases. Where the test and control nucleic acid duplexes contain one or more mismatched or unmatched bases, it will be understood that it may be necessary to increase the amount of oxidising or reactive agent since the mismatched or unmatched base may also be reactive to the oxidising or reactive agent.

As used herein, the term “single stranded nucleic acid molecule” is taken to refer to a single stranded molecule comprising at least two nucleotides, ie, a nucleic acid duplex has at least 2 pairs of nucleotides, preferably at least 10 pairs. Thus the methods of the invention may be applied to duplexes derived from hybridized single stranded nucleic acid molecules having from 2 nucleotides (ie a duplex having 2 pairs of nucleotides) up to whole genomes. Preferably the nucleic acid molecule is a nucleotide sequence.

As used herein, the term “nucleotide” is taken to refer to the monomeric unit which comprises a phosphate group, a sugar moiety, or modified sugar moiety, and a nitrogenous base. Preferred sugar moieties are the pentose sugars, such as ribose and deoxyribose, however, hexose sugars are also to be considered within the scope of the term “sugar moiety”. Modified sugar moieties include sugar moieties wherein the number and/or location and/or orientation of one or more hydroxyl groups has been altered from that found in naturally occurring sugar moieties, and/or where an oxygen atom has been replaced by another atom such as nitrogen or sulfur. The nitrogenous base is taken to refer to any nitrogen containing moiety which can act in pairing or mispairing in a nucleic acid duplex and as a proton acceptor. Preferred nitrogenous bases are cyclic, comprising preferably of one or more rings (e.g. mono- or bi-cyclic) and contain at least one nitrogen atom. Preferred nitrogenous bases include the pyrimidine bases such as uracil, thymine and cytosine, and the purine bases such as adenine and guanine or simple derivatives thereof such as deazapurines and inosine.

As used herein, a “nucleotide sequence” is taken to refer to a linear sequence of nucleotides selected from:

| | |
|---|---|
| 2′-Deoxycytidine 5′-phosphate; | Cytidine 5′-phosphate; |
| 2′-Deoxyadenosine 5′-phosphate; | Adenosine 5′-phosphate; |
| 2′-Deoxyguanosine 5′-phosphate; | Guanosine 5′-phosphate; |
| 2′-Deoxythymidine 5′-phosphate; | Thymidine 5′-phosphate; |
| 2′-Deoxyuridine 5′-phosphate; | Uridine 5′-phosphate |

or simple derivatives thereof such as deazapurines and inosine.

A “test nucleic acid duplex” includes a duplex which has been exposed to environmental or chemical conditions (test conditions), such as those described herein, such that it is desirable to test for conformational change as a result of those conditions. Of course, it will be appreciated that a test duplex may not actually have been exposed to conformational changing conditions but that it is nevertheless desirable to determine if it has or has not been exposed.

A “control nucleic acid duplex” includes a duplex having substantially the same (preferably identical) sequences of nucleotides and base pairing as the test nucleic acid duplex but whose conformation has not been changed by exposure to conformational changing conditions. It will, however, be recognised that a control nucleic acid duplex also includes a duplex which may contain conformational changes as a result of predetermined or quantified exposure to conformational changing conditions. Thus by providing a control duplex with a predetermined or quantified extent of conformational change the extent of conformational change in a test nucleic acid duplex can be compared.

As used herein, “conformational changing conditions” is intended to refer to conditions which result in a change of conformation or conformational change, in the nucleic acid duplex exposed to those conditions. Such conditions include environmental conditions such as radiation (eg electromagnetic or ionising), temperature change, pH change, electric current, magnetic field or change in salt concentration, and chemical conditions, ie exposure to chemical compounds such as enzymes and other compounds described herein. “Test conditions” refer to conformational changing conditions the effect of which are being tested for.

The nucleic acid duplexes may be extracted from natural sources or may be obtained commercially, synthetically or obtained from nucleic acid duplexes by melting and re-annealing and may be derived from purified genomic DNA or RNA, or PCR products. Hybridisation of the first and second single stranded nucleic acid molecules to form nucleic acid duplexes can be performed using methods known in the art or may occur as the result of the PCR process when amplifying a nucleic acid duplex. One suitable type of duplex is locked DNA which may allow reaction at higher temperatures and reduce oxidation or reaction due to melting.

It has been found that perturbed bases in a duplex may be selectively reactive towards an oxidising agent compared to a non-perturbed nucleotide base. Suitable oxidising agents for use in the present invention may include KMnO₄, OsO₄, chromic acid, ozone gas, peroxides (eg H₂O₂), perbenzoic acids (eg m-chloroperbenzoic acid and derivatives thereof), electrical current, etc. It is also possible in the invention to react perturbed bases with other reactive agents, other than oxidising agents, the reaction of which can be detected in a similar manner. An example of this type of reactive agent is carbodiimide.

An oxidising or reactive effective amount of an oxidising or reactive agent is an amount of the agent sufficient to modify or react (especially oxidise) a perturbed base or bases to the extent that the consumption of one or more starting agents or the formation of one or more products can be detected.

A “starting agent” is an agent (such as the oxidising or reactive agent or the first or test nucleic acid duplex) which is used in the oxidising or other reaction of the nucleic acid duplex under consideration. A “reaction product” is a product formed as a result of the oxidation or other reaction of a perturbed base in the duplex, such as the oxidised or otherwise reacted nucleic acid duplex or the reduced or reacted form of the starting agent.

Nucleic acid molecules can be either end-labelled or unlabelled. By use of a labelled (either end labelled or internally labelled) DNA or RNA as appropriate, it may be possible to obtain information about the location of perturbation. Any convenient label may be used, including, eg. radioactive labels, fluorescent labels and enzyme labels in a manner well known to those skilled in the art. Suitable labels include: ³²P, ³³P, 14C, FAM, TET, TAMRA, FLUORESCEIN, and JOE.

The oxidization of the nucleic acid duplex can be performed with all the starting agents in solution or by immobilising the duplex, or oxidizing or reactive agent, onto a solid support matrix. In certain embodiments of the invention, immobilising the duplex onto a solid support may be advantageous as it allows for the ready separation of the duplex from reaction solution and may thus simplify the detection of starting agents and/or reaction products. Suitable solid supports may be made of an appropriate polymeric material, be silicon derived (eg silica/glass) or paper. Supports may be in the form of pins, wells, plates or beads and may have a magnetic component or may be fully or partially coated with streptavidin so as to allow for attachment with a biotinylated duplex. In immobilising the duplex to the solid support, this may be done by attaching the duplex to the support, or alternatively, attaching a first single stranded nucleic acid molecule to the support and then hybridising a second single stranded nucleic acid molecule to it to form the attached duplex.

Determination of the presence of starting agents and/or reaction products can be carried out by any suitable means which may include spectroscopic (eg UV visible, NMR, mass or fluorescence spectrometry), chromatography (eg HPLC, GC), titration, colorimetry inorganic assay for the detection of oxidising agent or reduced form thereof (eg MnO₂) and electrochemical detection wherein a change in electrical current is indicative of a redox reaction. The oxidised or otherwise reacted nucleic acid duplex may also be detected by coupling the oxidised or reacted perturbed base to another organic molecule (eg an aldehyde) or another redox reagent system eg a redox stain, and detecting the formation of the resulting coupled product by a suitable means, for example as described above. In certain embodiments of the invention it may be useful to determine the presence of the oxidising agent and/or the reduced form of the oxidising agent.

In other embodiments of the invention, the formation of an oxidised or reacted duplex and/or the consumption of the starting duplex can be determined or detected by methods relying on melting temperature, for example by comparing the difference between the melting temperature of an oxidised or reacted duplex and the starting duplex or corresponding control duplex.

The melting temperature of the duplex is likely to be greatly decreased by the presence of an oxidised or reacted base over the presence of a perturbed unoxidised base and particularly over the unperturbed duplex. Thus, in another aspect of the invention, the oxidation or reaction methods described herein can be used to enhance existing techniques, ie separation techniques, for detecting a conformational change in nucleic acid duplex. Thus, in other embodiments of the invention, the formation of an oxidised or reacted test duplex and/or the consumption of the starting test duplex can be determined or detected by methods relying on melting temperature, for example by comparing the difference between the melting temperature of an oxidised or reacted test duplex and the starting test duplex or corresponding control duplex. In such embodiments of the invention, detection of a mismatched or unmatched base by oxidation methods (such as using KMnO₄ as described herein) can be used in conjunction with an increasing temperature gradient (such as about 2° C./minute). Thus the oxidation or reaction method is enhanced by the differential melting temperatures between a control duplex and a test duplex containing the perturbed base, wherein the test duplex has a lower initial melting temperature and therefore becomes more susceptible to oxidation or reaction by the oxidising or reactive agent. The reacted perturbed bases have the effect of further reducing the melting temperature of the test duplex, accentuating the difference in melting temperatures of the test duplex and control duplex.

The melting temperature of DNA duplexes can be readily measured with modern technology by straight absorbance or by adding a double stranded specific dye (eg. Syber green 1) to the oxidised duplex and unperturbed duplex and gradually increasing the temperature. As more and more single stranded DNA is produced the fluorescence is decreased which can be readily detected and the difference shown. Use of a single strand specific dye will also show the melting curve.

Suitable methods include Conformation Selective Gel Electrophoresis (CSGE), Denaturing Gradient Gel Electrophoresis (DGGE) or denaturing High Pressure Liquid Chromatography (dHPLC), wherein their discrimination is likely to be enhanced by the oxidative or reactive process.

Methods such as CSGE, dHPLC or DGGE rely on the discrepancy in melting temperature between a duplex and corresponding perturbed duplex. However, in certain instances, this discrepancy in melting temperature may not be sufficient to be adequately resolved and indicate the presence of a conformational change. However, an oxidised or reacted duplex, wherein a perturbed base has been oxidised by or reacted with an oxidising or reactive agent, would be expected to melt at a lower temperature than that of the unoxidised or unreacted duplex, thus providing a greater difference in melting temperature compared to the unperturbed duplex. This greater difference may aid in resolution, thus making “melting temperature” techniques more useful in identifying duplexes which contain a conformational change.

In the case of the process of DGGE where increasing denaturant (heat) pressure is applied, it is expected that the physical event on which the method relies can be detected without separation of the oxidised or reacted duplex, unoxidised or unreacted duplex and unperturbed duplex. Thus in this method a sudden opening of the duplex occurs during a slowly increasing temperature or denaturing concentration during electrophoretic separation. This opening will happen sooner in the perturbed duplex and is expected to occur even earlier after duplex oxidation or reaction. The unperturbed duplex opens later and moves further. The perturbed duplex is thus detected. If one slowly increases the denaturant (eg temperature, or chemical denaturant) in a tube in the presence of oxidising or reactive agent it would be expected to be able to detect a sudden increase in consumption of oxidising or reactive agent (or formation of product) when the duplex opens, it opening earlier in the case of the tube containing a perturbed duplex.

Another method of detecting the perturbed base is by use of allele specific oligonucleotide hybridisation which can be carried out on chips, beads, pins, wells etc or in liquid phase. Thus, the temperature at which the oxidised or reacted perturbed duplex will melt and hybridise with another piece of DNA (eg a probe) will be lower than that for the corresponding unoxidised or unreacted perturbed duplex, thereby potentially providing a greater differential hybridisation signal, and allowing for easier detection of a perturbed base.

Other separation methods which may be enhanced by the oxidative or reactive processes described herein include SSCP and sequencing, being methods known in the art. Agarose gels may be used to detect reaction products. The methods of the invention may be further used in conjunction with other reagents that react with perturbed bases such as hydroxylamine or carbodiimide. Thus, such reagents may show enhanced reactivity with a perturbed base after the perturbed base has been reacted with the oxidising or reactive agent (eg KMnO₄ or carbodiimide). Alternatively, oxidation or reaction of the perturbed base may be enhanced by firstly reacting the perturbed base with the reagent. Suitable conditions for reaction of hydroxylamine or carbodiimide with perturbed bases are described in, for example, EP 329 311 and Novack et al, PNAS, 83, 586-60 respectively. Other reagents may include enzymes such as repair enzymes (eg mut Y, mut A, excision nucleases, s1 nuclease and resolvases).

The rate of modification of the perturbed base depends on the nature of the base itself. Certain oxidising reagents (eg KMnO₄, OSO₄) are more selective towards thymine and uracil while the rate of the reaction with cytosine is slower. Rates of reaction are generally lower still where the perturbed base is guanine or adenine. Thus, preferably, the perturbed base to be modified is thymine, uracil or cytosine. Preferably where there are two complementary pairs of perturbed bases, these will include thymine (or uracil) and cytosine as this may allow for the detection of all conformational changes and give each change two chances of detection. Neighbouring bases may also be reactive due to local perturbation.

In a further embodiment of the invention, a conformational difference between two nucleic acid duplexes can be detected by carrying out the modification at a temperature just below the melting temperature of the perturbed duplex. Thus, when both a perturbed duplex and an unperturbed duplex are reacted with an oxidising or reactive agent at a temperature just below the melting temperature of the perturbed duplex, an oxidised or reacted perturbed duplex so formed will melt thus exposing T & C bases. This will result in a “burst” of oxidisation or reaction activity for the perturbed duplex which can be monitored by techniques described herein, eg by MnO₂ formation or KMnO₄ consumption.

The oxidising or other reaction for detecting a conformational change between a test nucleic acid duplex or sample and a control nucleic acid duplex or sample can be carried out in the range of about 0° C. to the melting point of the duplex, such as about 10-50° C. Preferably the oxidation or other reaction is performed in the temperature range of about 20-40° C., more preferably at about 25° C. or 37° C. The oxidation or other reaction can also be carried out above the melting point of the duplex eg up to about 80° C. by comparing to oxidative or reactivity rates such as due to differing numbers of T or C bases in each duplex.

The time taken for the oxidation or reaction may be dependent on the reaction temperature and the nature of the base to be modified. Preferably the time is in the range of about 1 minute to about 10 hours, eg. from about 5 minutes to about 3-4 hours. Preferably the modification is performed for about 10 minutes to about 1 hour, eg. about 30 minutes.

The modification is suitably carried out in aqueous solution or a mixture of aqueous and non-aqueous solvents, may be performed under acidic, neutral or basic conditions, and may optionally be performed in the presence of other agents such as a buffer, eg citrate or phosphate buffer. The modification can be carried out in the presence or absence of an amino base or salt thereof. Where a base is present, suitable amino bases may include alkyl amines (mono- and di-) and suitable salts thereof include sulfates, nitrates and halide salts, for example chloride. Examples of bases include tetraethylamine, tetram ethylamine diisopropylamine, tetraethylene diamine hydrazine and pyridine. Examples of preferred ammonium salts include tetraethylammonium chloride (TEAC) and tetramethylammonium chloride (TMAC). The base (or salt) solution may be of a concentration of between about 0 to about 6 M, preferably about 2-4 M, particularly about 3M.

In one preferred embodiment of the invention, the oxidising agent is KMnO₄. Permanganate oxidation (modification) of a perturbed nucleotide base (such as thymine) results in the formation of an unstable intermediate cyclic permanganate diester which decomposes under basic conditions to release the diol and soluble MnO₂.

MnO₂ absorbs strongly at 420 nm whereas MnO₄⁻ is almost transparent at this wavelength. However, MnO₄⁻ exhibits strong absorption at 525 nm. Thus, conveniently, the oxidation reaction can be monitored by UV spectroscopy at a wavelength of in the range of about 400-440 nm, more preferably in the range of 410-430 nm such as about 420 nm for the formation of MnO₂ and/or in the range of about 505-545 nm, more preferably in the range of 515-535 nm such as about 525 nm for the consumption of KMnO₄.

Preferably the KMnO₄ is used in a molar excess per perturbed base, for example at least about 3 molar excess, more preferably about 5 molar excess, if the formation of MnO₂ is being detected. If the consumption of KMnO₄ (MnO₄ is preferably used in an approximately equimolar amount per perturbed base.

In a preferred embodiment, a perturbed T base, U base or C base is modified by KMnO₄.

Since the two manganese species both give strong absorption in the visible region, determination of the presence of MnO₄⁻ or MnO₂ can also be carried out by simple visual analysis, for example, MnO₄— exhibits a pink colour in TEAC while MnO₂ exhibits a yellow colour in TEAC.

The presence of a perturbed base can also be determined by comparison of the respective isosbestic points for a test (eg perturbed) duplex and its corresponding controlled (eg unperturbed) duplex. The isosbestic point in an absorption spectrum of two substances (eg. MnO₂ and MnO₄—) in equilibrium with each other is the wavelength at which the two substances have the same molar extinction coefficients. By sequential scanning over a suitable time interval in the UV-visible region, the isosbestic point for the conformational modification of a nucleic duplex acid sample can be determined. Unperturbed nucleotide bases in a duplex react more slowly than perturbed bases. Thus, after a predetermined interval, the isosbestic point for a perturbed duplex would be expected to be different to that of an unperturbed duplex. The isosbestic point can be used in combination with the rate of change of absorbance to obtain more accurate determinations.

A relative comparison of the isosbestic point for two nucleic acid duplexes can also be used to detect a difference in conformation between two nucleic acid duplexes which may have been exposed to different environmental and/or chemical conditions. Oxidative methods for detecting the difference between two such nucleic acid duplexes can be performed as described herein.

The invention may also be particularly applicable to screen multiple samples in a high throughput fashion. This aspect of the invention is particularly applicable for example in the situation of screening chemical compounds, such as potential pharmaceutical agents, for mutagenic, carcinogenic, anticancer or antimicrobial (especially antibiotic) activity.

In a further aspect of the invention, components and/or reagents for performing the present invention may be presented in a kit. The kit can be provided in compartmentalised form adapted for use in the present invention and may include one or more of: oxidising or reactive agent, base (or salt thereof), test or control nucleic acid duplexes (eg calf thymus), buffers, spectroscopic cells and solid support phases, and may further be provided with instructions for performing the invention.

The method of the present invention is particularly useful for the testing of DNA obtained from mammalian cells, (eg. human; simian; livestock animals such as cows, goats, sheep, horses, pigs; laboratory test animals such as rats, mice, guinea pigs, rabbits; domestic companion animals such as dogs, cats; or captive wild animals), fish cells, reptile cells, bird cells, insect cells, fungi cells, bacterial cells or viral agents, parasitic agents, (eg. Plasmodium, Chiamydia, Rickettsia and protozoa) and plant cells including tobacco, ornamental trees, shrubs and flowering plants (eg. roses), trees, plants which product fruits and vegetables for human or animal consumption (eg. apples; pears; bananas; citrus fruit; stone fruit, including peaches, cherries, plums; potatoes; root vegetables; cabbage family etc) and agricultural crops such as oats, corn, barley, rye, cotton, sunflower, wheat, rice and legumes such as peas and soya, and laboratory test plants such as Aribidopsis thalniana.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

The present invention will now be described further, with reference to the following examples, which are provided for the purpose of illustrating certain embodiments of the invention and are not intended to limit the generality hereinbefore described.

### EXAMPLES

Chemicals and Spectrophotometer:

Chemicals, solvents and calf thymus DNA were purchased from Aldrich Chemical Company (Castle Hill, Australia).

Oxidation reactions of DNA with potassium permanganate were carried out in glass 1.2 ml quartz cuvette and the spectral data were obtained from Cintra-10 spectrophotometer (GBC Scientific Equipment Pty Ltd, Victoria, Australia) or Cary 300 UV-Visible spectrophotometer (Varian Inc., Victoria, Australia) by recording the absorbance vs. time curves at pre-selected wavelengths and/or by repetitive scanning of the ultraviolet-visible region (200 to 800 run).

Preparation of calf thymus Z-DNA samples: Z-DNA was prepared by treatment of the commercially available calf thymus DNA with 3M NaCl solution.

### Example 1

### Oxidation Reaction of B and Z-Calf Thymus DNA Samples

Calf thymus DNA (5 μl, 8.27 μg) samples were incubated with 965 μl of 3M aqueous NaCl solution at 40° C. for 1 h. The resulting mixture (Z-DNA) was treated with 30 μl of 10 mM KMnO₄ solution. The mixture was immediately transferred to 1.2 ml quartz cuvette and then scanned from 200 to 800 nm at 25° C. (every 10 min) over 120 min. In the control experiment (without NaCl), the calf thymus B-DNA (5 μl, 8.27 μg) was dissolved in 965 μl of distilled H₂O and the treated under identical reaction conditions as described above. The results are tabulated in Table 1.

TABLE 1
The Oxidation Method for Detection of DNA Conformational
Changes Induced by Inorganic Substances

| | Level of | Level of | |
|---|---|---|---|
| | oxidation | oxidation | |
| Experiments | (2 min) | (2 h) | Isosbestic point |
| B-DNA + KMnO₄ | 0.030 | 0.097 | 489 nm |
| Z-DNA + KMnO₄ | 0.057 | 0.227 | 493 nm |

B-DNA (calf thymus DNA); Z-DNA (calf thymus DNA in 3 M NaCl)
Level of oxidation (arbitrary unit) was based on the absorbance at 420 nm
All experiments were carried out at 25° C. with the Cintra-10 Spectrophotometer.

Physical and chemical properties of the perfect DNA have been well documented under normal conditions (low ionic strength and at physiological pH). The DNA conformational change can be dramatically enhanced when the DNA samples are converted into Z-form DNA by using high salt concentrations. Under a specific condition the chemical and physical properties of unwound DNA are changed due to different degree of exposure of nucleotide bases and the Z-duplex DNA became hyperactive towards chemical reactions compared to the B-DNA. The result of the experiment indicated that the double stranded B-DNA is quite unreactive towards KMnO₄ compared to the Z-conformer, which became highly susceptible to the reaction with permanganate (Table 1). Both B- and Z-DNA conformers were clearly distinguished by using the patterns of oxidation spectra and the isobestic points.

### Example 2

### Reaction of Calf Thymus DNA With Intercalators

Calf thymus DNA (511, 8.27 μg) was mixed with ethidium bromide (10 μl, 6.3 mmol) in distilled H₂O (950 μl). The mixture was gently mixed on a shaker at 40° C. for 1 h. After incubation, KMnO₄ (35 μl of 10 mM solution) was added to the reaction mixture and the resulting mixture was transferred to the quartz cuvette and the sample was scanned from 200 to 800 nm at 25° C. (every 10 min) over 120 min. The level of oxidation induced by intercalator is based on the net absorbance (measured at 420 nm) and calculated as follows:

Net A420 nm=[A420 nm of the Expt (120 min)−A420 nm of the Expt (2 min)]−[A420 nm of the Control (120 min)−A420 mm of the Control (2 min)]

Where Net A420 run represents the level of oxidation of the DNA-chemical adduct.

The Expt=calf thymus DNA+DNA binding agent+KMnO₄

The Control=DNA binding agent+KMnO₄.

For the control experiment, calf thymus DNA (5 μl, 8.27 μg) was mixed with glucose (10 μl, 6.3 nmol) in distilled H₂O (950 μl). The reaction was carried out under identical conditions as described above. The results were tabulated in the Table 2

TABLE 2
Permanganate Oxidation of Calf Thymus DNA Induced by
Intercalators

| | | | Level of |
|---|---|---|---|
| | | | oxidation |
| | ΔA420 nm | ΔA420 nm | (induced |
| | (2 min-2 h) | (2 min-2 h) | by chemical) |
| Experiments | (Expt) | Control | Net A420 nm |
| DNA | 0.110 | — | 0.110 |
| DNA + glucose | 0.1283 | 0.010 | 0.118 |
| DNA + ethidium bromide | 0.333 | 0.140 | 0.193 |

Level of oxidation (arbitrary unit) was based on the net absorbance at 420 nm
All experiments were carried out at 35° C. with the Cary 300 Spectrophotometer.

Effect of the Ethidium Bromide Intercalator:

Ethidium bromide is well known as non-sequence specific intercalator. When the molecule sandwiches between nucleotide bases it unwinds the duplex by 28°. The unwinding effect dramatically increased the exposure of nucleotide bases and thus facilitates the oxidation process. The results confirmed that the method can be used to identify the DNA binding agents as the control experiment (DNA+glucose) showed no increase of the oxidation level during 2-hour incubation (Table 2).

Ethidium bromide is also reported to give the reverse effect on Z-DNA by relaxing the “supercoil” property of the Z-conformation to the B-conformation. As a consequence, the oxidation level was decreased when the intercalator was added to the Z-DNA solution. To demonstrate the effect of the DNA binding agents on B- and Z-conformers of the calf thymus DNA, two following experiments were carried out:

(i) Effect of intercalator on Z-DNA—application for determination of DNA unwinding degree; and

(ii) Effect of the concentration of intercalator on B-DNA.

(i) Effect of intercalator on Z-DNA—Application for determination of unwinding degree:

Due to limited information about the degree of unwinding of intercalators, only four intercalators with different degree of unwinding were employed in the experiment: doxorubicin (10°), daunorubicin (12°), 9-aminoacridine (17°) and ethidium bromide (28°). The Z-DNA samples were mixed in equal amounts of intercalators and the reactions were carried out under identical conditions. Calf thymus DNA (5 μl, 8.27 μg) was incubated with NaCl (600 μl, SM NaCl solution), the DNA binding agent or intercalator (10 μl, 6.3 nmol) in distilled H₂O (355 μl). 4 non-sequence specific intercalators were used in the model study: doxorubicin, daunorubicin, 9-aminoacridine and ethidium bromide. The reaction mixture was gently mixed on shaker at 40° C. for 1 h. After incubation time, KMnO₄ (30 μl) was added to the reaction mixture and the resulting mixture was transferred to the quartz cuvette and the absorbance (420 nm) was obtained at 35° C. for 2- and 60-min time-points. The level of oxidation induced by intercalator is based on the net absorbance and calculated as follows:

Net A420 nm=[A420 nm of the Expt (60 min)−A420 nm of the Expt (2 min)]−[A420 nm of the Control (60 min)−A420 nm of the Control (2 min)]

Where Net A420 nm represents the level of oxidation of the DNA-chemical adduct.

The Expt=calf thymus DNA+NaCl+DNA binding agent+KMnO₄

The Control=DNA binding agent+KMnO₄.

The control experiment (without DNA binding agent) was treated under identical conditions as described above.

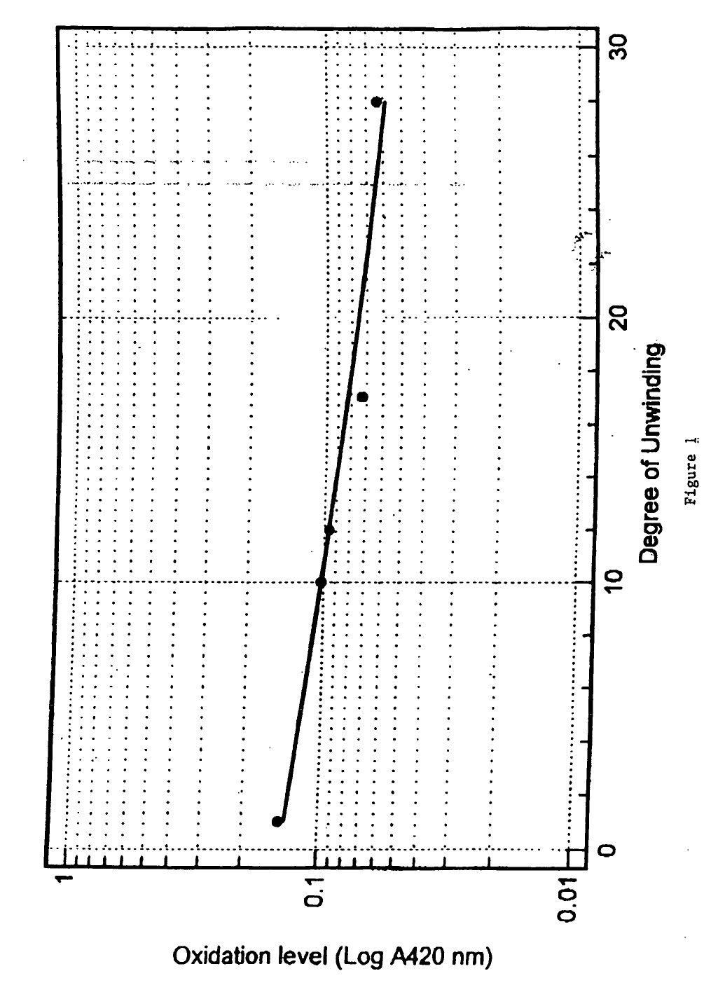

Kinetic data confirmed that the oxidation was slowest when DNA sample was incubated with ethidium bromide and fastest with doxorubicin (Table 3). After a one-hour oxidation process, the correlation between the oxidation level and the degree of unwinding is a linear curve with high correlation coefficient (0.94). The correlation between level of oxidation and degree of unwinding of the calf thymus DNA sample was plotted in the linear graph (FIG. 1). The regression equation, correlation coefficient and other statistical data (mean and variance) was calculated by using the MedCalc software. The published data associated with the degree of unwinding has been used in this study: doxorubicin (10°), daunorubicin (12°), 9-aminoacridine (17°) and ethidium bromide (28°) (FIG. 1). The control sample (without intercalator) was used as a reference for an initial degree of Z-DNA. For the purpose of calculation of log value, 1 degree was assigned to the initial degree of Z-DNA. It would be envisaged that this correlation curve acts as a standard curve to predict the degree of unwinding for any other substances assuming equal mole per mole binding for each intercalator with DNA.

TABLE 3
Permanganate Oxidation of Calf Thymus DNA Induced
by Intercalators

| | | | Level of |
|---|---|---|---|
| | | | oxidation |
| | ΔA420 nm | ΔA420 nm | (induced by |
| | (2 min-1 h) | (2 min-1 h) | chemical) |
| Experiments | (Expt) | (Control) | Net A420 nm |
| DNA + NaCl | 0.1426 | — | 0.1426 |
| DNA + NaCl + | 0.1427 | 0.0406 | 0.1029 |
| doxorubicin | | | |
| DNA + NaCl + | 0.1670 | 0.0713 | 0.0957 |
| daunorubicin | | | |
| DNA + NaCl + 9- | 0.1327 | 0.0601 | 0.0726 |
| aminoacridine | | | |
| DNA + NaCl + ethidium | 0.1761 | 0.1128 | 0.0633 |
| bromide | | | |

Level of oxidation (arbitrary unit) was based on the net absorbance at 420 nm
All experiments were carried out at 35° C. with the Cary 300 Spectrophotometer.

(ii) Effect of Concentrations of the Intercalator:

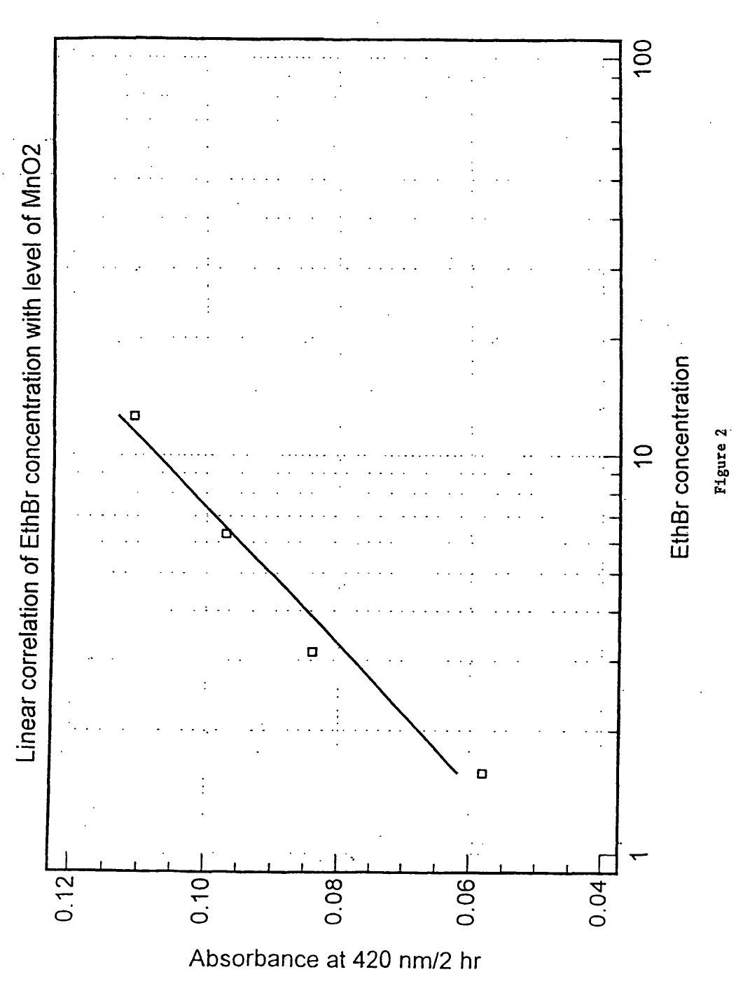

The effect of concentration of one intercalator, ethidium bromide is summarised in Table 4. DNA calf thymus (5 μl, 8.27 μg) was incubated with 4 different concentrations of ethidium bromide (1.57 nmol, 3.15 mmol, 6.3 mmol and 12.6 nmol). All tubes {4 experiment and 4 control samples (without DNA)} were gently mixed on a shaker at 40° C. for 60 min. After incubation the mixtures were treated with 35 μl of 10 mM KMnO₄ and immediately transferred to cuvettes for measurement of the absorbances at 420 nm at 2 min and 120 min. The level of oxidation obtained from each sample was calculated as described above and the results were plotted as a linear graph (Table 4 & FIG. 2). All experiments were carried out in duplicate. The result indicated that the level of oxidation was strongly dependent on the concentration of ethidium bromide. At increasing concentrations of ethidium, the number of intercalator binding sites along the DNA sequence increased and therefore the level of oxidation is highly proportional to the concentration of ethidium bromide within the range of the experiment. The standard curve (concentrations of ethidium bromide vs level of oxidation) is linear with a very high correlation coefficient (0.97). When the concentration exceeded this limit, the curve becomes flat due to saturation of intercalator binding (results not shown).

TABLE 4
Permanganate Oxidation of Calf Thymus DNA Induced by
Different Concentrations of Ethidium Bromide.

| | ΔA420 nm | ΔA420 nm | Level of oxidation |
|---|---|---|---|
| Concentrations of | (2 min-2 h) | (2 min-2 h) | (induced by chemical) |
| ethidium bromide | (Expt) | (Control) | Net A420 nm |
| 1.575 nmol | 0.125 | 0.067 | 0.058 |
| 3.15 nmol | 0.151 | 0.067 | 0.084 |
| 6.3 nmol | 0.228 | 0.131 | 0.097 |
| 12.6 nmol | 0.295 | 0.184 | 0.111 |

Level of oxidation (arbitrary unit) was based on the net absorbance at 420 nm
All experiments were carried out at 25° C. with the Cintra-10 Spectrophotometer.

### REFERENCES

1. Herbert, A. and Rich, A. (1999). Left-handed Z-DNA: structure and function. Genetica, 106, 37-47.

2. Jeppesen, C. and Nielsen, P. E. (1988). Detection of intercalation-induced changes in DNA structure by reaction with diethyl pyrocarbonate or potassium permanganate. FEBS LETTERS, 231, 172-176.

3. Coury, J. E., McFail-Isom, L., Williams, L. D., and Bottomley, L. A. (1996). A novel assay for drug-DNA binding mode, affinity and exclusion number: scanning force microscopy. Proc. Natl Acad. Sci. USA, 93, 12283-12286.

1. A method of detecting a conformational change in a test nucleic acid duplex sample, which has been exposed to test conditions, relative to a control nucleic acid duplex sample, which comprises the steps of separately subjecting the test and control duplex samples to treatment with an oxidising or reactive effective amount of an oxidising or reactive agent for a time and under conditions sufficient to oxidise or otherwise react with perturbed bases within the duplexes, and determining if there is a difference in:
(a) formation or rate of formation of one or more reaction products; and/or
(b) consumption or rate of consumption of one or more starting agents;
between test sample and control sample, wherein a difference indicates conformational change of the test sample.

2. A method of determining extent of conformational change in a nucleic acid duplex sample, which has been exposed to test conditions, relative to a control nucleic acid duplex sample, which comprises the steps of separately subjecting the samples to treatment with an oxidising or reactive effective amount of an oxidising or reactive agent for a time and under conditions sufficient to oxidise or otherwise react perturbed bases within the duplexes, and quantifying difference in:
(a) formation or rate of formation of one or more reaction products of oxidisation or reaction; and/or
(b) consumption or rate of consumption of one or more starting agents of oxidisation or reaction;
between test sample and control sample and comparing the difference to quantified differences determined from exposing like nucleic acid duplex samples from testing with agents that give rise to a known extent of conformational change.

3. A method of detecting a conformational difference between two nucleic acid duplex samples which comprises the steps of separately subjecting a first and a second nucleic acid duplex sample to treatment with an oxidising or reactive effective amount of an oxidising or reactive agent for a time and under conditions sufficient to oxidise or otherwise react with perturbed bases with the duplexes and determining if there is a difference in:
(c) formation, or rate of formation, of one or more reaction products;
(d) consumption, or rate of consumption, of one or more starting agents;
between the first and second nucleic acid duplex samples wherein a difference is indicative of a conformational difference between the two nucleic acid duplex samples.

4. A method of determining whether a test nucleic acid duplex has been exposed to conformational changing conditions comprising the steps of separately subjecting the test nucleic acid duplex sample and a control nucleic acid duplex sample to treatment with an oxidising or reactive effective amount of an oxidising or reactive agent for a time and under conditions sufficient to oxidise or otherwise react with perturbed bases within the duplexes, and determining if there is a difference in:
(a) formation or rate of formation of one or more reaction products; and/or
(b) consumption or rate of consumption of one or more starting agents;
between the test duplex sample and control duplex sample, wherein a difference indicates exposure of the test sample to conformational changing conditions.

5. The method according to any one of claims 1-4 wherein the monitoring of the formation, or rate of formation, of one or more reaction products and/or the consumption, or rate of consumption, of one or more starting agents is monitored by a technique selected from the group consisting of UV visible spectroscopy, fluorescence spectroscopy, NMR spectroscopy, mass spectroscopy, chromatography, titration, colorimetry, electrochemical detection, visual detection, melting temperature detection and redox stain.

6. The method according to claim 5 wherein the formation, or rate of formation, of one or more reaction products and/or the consumption, or rate of consumption, of one or more starting agents involves monitoring for the presence of the oxidising agent and/or the reduced form of the oxidising agent.

7. The method according to claim 5 wherein the technique is UV visible or fluorescence spectroscopy.

8. The method according to claim 7 wherein the oxidising agent is KMnO₄ and the monitoring of the formation, or rate of formation, of a reaction product involves measuring absorbance at about 420 nm.

9. The method according to claim 8 wherein the oxidising agent is KMnO₄ and the monitoring of the consumption, or rate of consumption, of a starting agent involves measuring absorbance at about 525 nm.

10. The method according to claim 9 further comprising the determination of the isosbestic point for the oxidation of each of the test and control nucleic acid duplexes.

11. The method according to claim 5 wherein the technique is visual detection.

12. The method according to claim 5 wherein the technique involves monitoring for the melting temperature of the duplex undergoing treatment with the oxidising agent.

13. The method according to any one of claims 1 to 4 wherein the oxidising agent is KMnO₄.

14. The method according to any one of claims 1, 2 or 4 wherein the test condition or conformational changing condition is an environmental condition selected from the group consisting of radiation, change in temperature, change in pH, electrical current, magnetic field, or change in salt concentration or exposure to a chemical compound selected from the group consisting of potential pharmaceutical or veterinary products, agrochemicals, products intended for human or animal consumption, cosmetics or other personal use products, compounds found in clothing or used in manufacturing processes or identified from sources such as plant, animal or microorganism extracts or soil, water or air samples.

15. A kit adapted for performing the method of any one of claims 1 to 4, wherein said kit is in compartmentalised form and comprises at least two components selected from the group consisting of oxidising or reactive agent, base (or salt thereof) test nucleic acid duplex or, control nucleic acid duplex, buffer and spectroscopic cell.
